(19) United States
(12) Patent Application Publication
(10) Pub. No.: US 2023/0298690 A1
(43) Pub. Date: Sep. 21, 2023

(54) GENETIC INFORMATION PROCESSING SYSTEM WITH UNBOUNDED-SAMPLE ANALYSIS MECHANISM AND METHOD OF OPERATION THEREOF

(71) Applicant: AIONCO, Inc., Menlo Park, CA (US)

(72) Inventors: Cheuk Ying Tang, Cupertino, CA (US); Victor Solovyev, San Francisco, CA (US); Sidney Tobias, Redwood City, CA (US); Gene Lee, San Mateo, CA (US)

(21) Appl. No.: 18/168,554

(22) Filed: Feb. 13, 2023

Related U.S. Application Data
(60) Provisional application No. 63/309,893, filed on Feb. 14, 2022.

(51) Int. Cl. G16B 20/00; G16B 30/10; G16B 40/20; G16H 50/20

(52) U.S. Cl. CPC G16B 20/00; G16B 30/10; G16B 40/20; G16H 50/20

(57) ABSTRACT

Introduced here is an approach to detect existence of cancer or a likely onset of cancer based on analyzing DNA data derived from unbounded samples that are not limited to specific locations of a patient’s body or specific types of cancers. One or more machine learning models may be developed using targeted patterns in the human genome. The machine learning models may be trained to analyze and detect mutation patterns characteristic of one or more cancers. The trained models may be used to analyze the unbounded samples to assess the existence cancer or the proximity to the onset of cancer based on identifying mutation patterns in the patient DNA to the patterns characteristic of the one or more cancers.

### CROSS-REFERENCE TO RELATED APPLICATIONS

This application claims priority to U.S. Provisional Application No. 63/309,893, titled “GENETIC INFORMATION PROCESSING SYSTEM WITH GENERAL-SAMPLE ANALYSIS MECHANISM AND METHOD OF OPERATION THEREOF” and filed on Feb. 14, 2022, which is incorporated by reference herein in its entirety.

### TECHNICAL FIELD

Various implementations concern computer programs and associated computer-implemented techniques for processing sequenced information, such as text-based representation of genetic information.

### BACKGROUND

Genes are pieces of deoxyribonucleic acid (DNA) inside cells that indicate how to make the proteins that the human body needs to function. At a high level, DNA serves as the genetic “blueprint” that governs operation of each cell. Genes can not only affect inherited traits that are passed from a parent to a child, but can also affect whether a person is likely to develop diseases like cancer. Changes in genes — also called “mutations” — can play an important role in the physiological conditions of the human body, such as in the development of cancer. Accordingly, genetic testing may be leveraged to detect such physiological conditions or likely onsets thereof.

The term “genetic testing” may be used to refer to the process by which the genes or portions of genes of a person are examined to identify mutations. There are many types of genetic tests, and new genetic tests are being developed at a rapid pace. While genetic testing can be employed in various contexts, it may be used to detect mutations that are known to be associated with cancer.

Genetic testing could also be employed as a means for addressing or treating the physiological condition. For example, after a person has been diagnosed with cancer, a healthcare professional may examine a sample of cells to look for changes in the genes in tracking the progress of the cancer, the treatment, etc. These changes may be indicative of the health of the person (and, more specifically, progression/regression of the cancer). Insights derived through genetic testing may provide information on the prognosis, for example, by indicating whether treatment has been helpful in addressing the mutation.

Implementing computing technologies for the genetic testing may yield valuable insights. For example, artificial intelligence and machine-learning technologies may be leveraged to analyze DNA information for detecting and/or addressing cancers or potential onset of cancers. However, the magnitude of the DNA information, the large number of potential mutations, large number of samples, and other similar factors often negatively impact the effectiveness, the accuracy, and the practicality in leveraging such computing technologies for the genetic testing.

### BRIEF DESCRIPTION OF THE DRAWINGS

FIGS. 1A and 1B show example operating environments of a computing system including a genetic information processing system (or simply “system”) that includes an unbounded sample analysis mechanism in accordance with one or more implementations of the present technology.

FIG. 2 shows an example data processing formats for the genetic information processing system in accordance with one or more implementations of the present technology.

FIG. 3 shows example expected phrases in accordance with one or more implementations of the present technology.

FIG. 4 shows example derived phrases in accordance with one or more implementations of the present technology.

FIG. 5 shows an example analysis template in accordance with one or more implementations of the present technology.

FIG. 6 shows an example control flow diagram illustrating the functions of the system in accordance with one or more implementations of the present technology.

FIGS. 7A and 7B show flow charts of example methods of operating a computing system in accordance with one or more implementations of the present technology.

FIG. 8 shows charts illustrating mutations detected in tumor samples and unbounded samples using the usable locations in accordance with one or more implementations of the present technology.

FIG. 9 shows a chart illustrating a matrix of likelihood values output by a model upon being applied to sample DNA information of an example set of patients.

FIG. 10 is a block diagram illustrating an example of a system in accordance with one or more implementations of the present technology.

Various features of the technology described herein will become more apparent to those skilled in the art from a study of the Detailed Description in conjunction with the drawings. Various implementations are depicted in the drawings for the purpose of illustration. However, those skilled in the art will recognize that alternative implementations may be employed without departing from the principles of the technology. Accordingly, although specific implementations are shown in the drawings, the technology is amenable to various modifications.

### DETAILED DESCRIPTION

Genetic testing may be beneficial for diagnosing and treating cancer. For example, identifying mutations that are indicative of cancer can help (1) healthcare professionals make appropriate decisions, (2) researchers to direct their investigations, and (3) precision medicine to design better therapies. However, discovering these mutations tends to be difficult, especially as the number of cancers of interest (and thus, corresponding data) increases.

While computer-aided detection (CADe) and computer-aided diagnostic (CADx) processing systems may be used to analyze the genetic testing data, conventional approaches still face several drawbacks due to the overwhelming number of computations required for such analysis. For example, conventional systems may identify a number of molecular positions (e.g., target analysis locations) and combinations that may inefficient, ineffective, inaccurate, or otherwise impractical to process. Moreover, such deficiencies become even more problematic when the system is tasked with reviewing the genetic information of tens, hundreds, or thousands of patients. In other words, even if a conventional system is able to comprehensively analyze the genetic information of a single patient, reviewing the genetic information of tens, hundreds, or thousands of patients during actual deployment becomes impractical due to the processing delays and inaccuracies.

Introduced here is an approach that can be implemented by a computing system to predict and/or diagnose one or more types of cancers in an improved manner. Implementations of the present technology can include the computing system processing the genetic information as relatively simple/smaller computer-readable data, such as text strings (simpler/smaller in comparison to, e.g., image data). Using the textual representations, the computing system can identify specific text patterns, such as unique segments of repeated characters (e.g., tandem repeats (TRs) corresponding to sequences of two or more DNA bases that are repeated numerous times in a head-to-tail manner on a chromosome), phrases surrounding the unique segments, and derivations/mutations thereof, used to analyze nucleic acid sequences (or simply “sequences”). In some implementations, the computing system can focus on the unique phrases and/or derivations thereof in characterizing and/or recognizing one or more types of cancer. In some implementations, the computation system can select features from the phrases/derivations and may ignore other portions of the overall text string or sequence, thereby reducing the overall computations in developing, training, and/or applying a machine learning (ML) model or other artificial intelligence mechanisms. While implementation of the approach may result in improvements across different aspects of mutation discovery, there are several notable improvements worth mentioning.

Advantageously, the approach allows models to be trained (and diagnoses to be predicted by those trained models) in a more time- and resource-efficient manner as the number of features considered by the computing system may be reduced (e.g., from tens of thousands of nucleotide locations to several thousand nucleotide locations). For a given type of cancer, the computing system can reduce an expanded feature set that is discovered through examination of training of genetic information through ML, so as to identify the most important nucleotide locations from a diagnostic perspective without significantly harming the accuracy in identifying mutations that are indicative of the given cancer type.

In some implementations, the computing system can include and/or utilize a mutation analysis mechanism that identifies a set of unique portions or segments in the human genome/DNA and related mutations that correspond to development/onset of certain types of cancer. The computing system can identify the set of unique portions or phrases and mutations (e.g., text strings having a length of k) based on the TRs.

The computing system can use the set of unique phrases and/or mutations to identify indicators (e.g., biomarkers) in unbounded samples (e.g., cells or biological components found in blood, saliva, or the like) that are not limited to specific cancers or specific regions (e.g., region directly affected by the targeted cancer) of the patient body. For example, the computing system can use the set of unique phrases and mutations (usable list of TRSs) to identify and detect patterns indicative of one or more types of cancers in the DNA information obtained from leukocyte or white blood cells.

Conventionally, the leukocyte DNA information has been used as comparison or normal data in contrast to cancerous or tumor DNA information. Previously, leukocyte DNA was understood as remaining steady and largely unaffected by cancer or corresponding genetic mutations.

It has been discovered that using the set of unique phrases and mutations to analyze the leukocyte DNA information (e.g., the textual representations thereof) identifies unique characteristics or patterns indicative of one or more types of cancers. For each type of cancer, the discovered characteristics/patterns and the corresponding mutations are different than the characteristics/patterns found in tumor samples. In other words, analysis of the leukocyte DNA information using the set of unique phrases and mutations has identified mutations in the leukocyte DNA that likely result from physiological interacting with the existing cancer or partially mutated cells that can indicate/predict likely onset of cancer in the near future (within a threshold duration). The characteristic patterns/mutations in the leukocyte DNA information can be used as features to develop and train leukocyte-based ML models that detect or predict onset of one or more types of cancer from patient’s blood sample.

Using the same approach, the set of unique phrases and mutations can be used to identify characteristics/patterns indicative of cancers (e.g., biomarkers) of DNA information derived from other unbounded samples. For example, unique characteristics/patterns can be identified in the DNA information derived or sequenced from the saliva samples or cheek swabs of patients with one or more types of cancers. Accordingly, the system can develop and train ML models that detect or predict onset of one or more types of cancer from the DNA information obtained from unbounded samples or samples collected from regions different than the regions affected by the one or more types of cancer.

Implementations may be described in the context of instructions that are executable by a system for the purpose of illustration. However, those skilled in the art will recognize that aspects of the technology described herein could be implemented via hardware, firmware, or software. As an example, a computer program that is representative of a software-implemented genetic information processing platform (or simply “processing platform”) designed to process genetic information may be executed by the processor of a system. This computer program may interface, directly or indirectly, with hardware, firmware, or other software implemented on the system. Moreover, this computer program may interface, directly or indirectly, with computing devices that are communicatively connected to the system. One example of a computing device is a network-accessible storage medium that is managed by a healthcare entity (e.g., a hospital system or diagnostic testing facility).

### Overview of Genetic Information Processing System

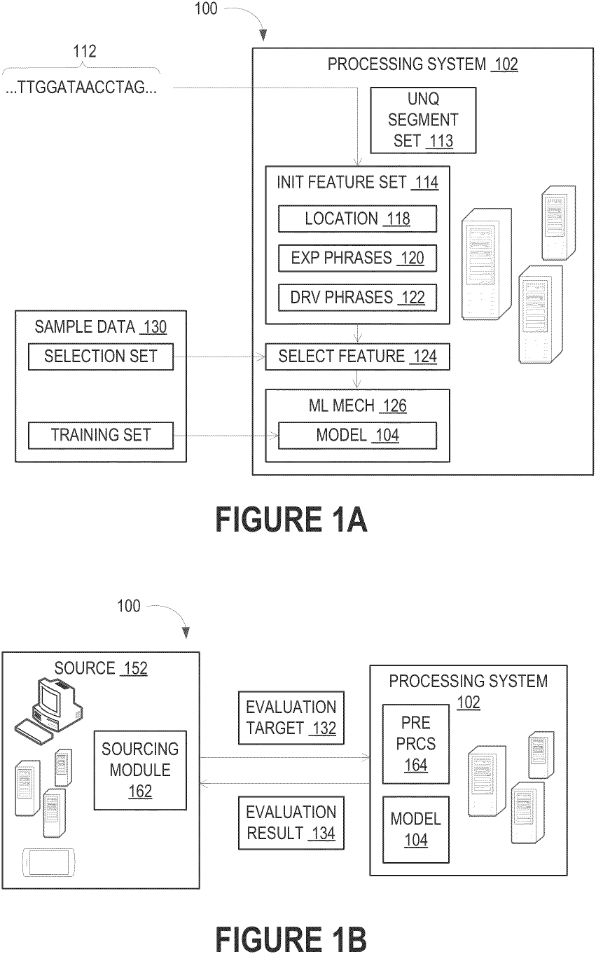

FIGS. 1A and 1B show example operating environments of a computing system 100 including a genetic information processing system 102 (“processing system 102”) in accordance with one or more implementations of the present technology. The processing system 102 can include one or more computing devices, such as servers, personal devices, enterprise computing systems, distributed computing systems, cloud computing systems, or the like. The processing system 102 can be configured to analyze DNA information for diagnosing one or more types of cancer, for evaluating development stages leading up to the onset of the one or more types of cancer, and/or for predicting a likely onset of the one or more types of cancer.

The application environment depicted in FIG. 1A can represent a development or training environment in which the processing system 102 develops and trains an analysis mechanism, such as a ML model 104, configured to detect a presence, a progress, and/or a likely onset of one or more types of cancer. In developing and training the ML model 104, the processing system 102 can first identify an analysis template (e.g., specific data locations or values within a reference data 112, such as the human genome or other data derived from human/patient DNA) targeted for further analysis/consideration. The reference data 112 can further include DNA information obtained or sequenced from one or more types of cancers/tumors, control samples (e.g., leukocytes), other unbounded samples, or both.

As an illustrative example, the processing system 102 can use a text-based representation (e.g., one or more text string using ‘T’, ‘A’, ‘G’, and ‘C’) of the human DNA as the reference data 112. The processing system 102 can analyze the reference data 112 to identify specific locations and/or corresponding text sequences that can be utilized as identifiers or comparison points in subsequent processing. In some implementations, the processing system 102 can use a set of unique text segments 113 (e.g., a set of unique TRs) found or expected in the reference data 112 to generate an initial feature set 114. The processing system 102 can generate the initial feature set 114 by identifying expected phrases that include the unique segment set 113 and/or by computing derivations thereof (e.g., derived phrases) that represent mutations targeted for analysis. The initial feature set 114 and/or the unique segment set 113 can include location identifiers 118 associated with a relative location of such segments, phrases, and/or derivations within the reference data 112. In some implementations, for each type of cancer, the initial features set 114 can identify text sequences of mutations that are unique or characteristic to the unbounded samples, such as leukocyte-based data or saliva-based data.

For the feature selection, the processing system 102 can iteratively add or remove one or more unique locations/sequences and/or derivations from the initial feature set 114 and calculate a correlation or an effect of the removed data point on duplicating the known classifications of the sample data 130 (e.g., to accurately recognize the different categories of the sample data 130). The processing system 102 can determine a set of selected features 124 that correspond to the unique locations/phrases and derivations thereof having at least a threshold amount of affect or correlation with one or more corresponding cancer types. In other words, the processing system 102 can determine the set of features 124 including locations, sequences, mutations or combinations thereof that are deterministic/characteristic of or commonly occurring in corresponding cancers. Based on the selected set of features 124, the processing system 102 can implement a ML mechanism 126 (e.g., random forest, neural network, logistic regression, etc.) to generate the ML model 104. The processing system 102 can further train the ML model 104 using training data.

Using the features (e.g., text segments/phrases), the processing system 102 can limit the amount of data considered or processed in subsequent analyses, such as in feature selection, model generation, model training, and/or the like. For example, the processing system 102 can use the targeted segments/phrases to reduce the size of analyzed data. Accordingly, the processing system 102 can reduce the resource consumption through the reduced size of the selected feature set.

The application environment depicted in FIG. 1B can represent a deployment environment in which the processing system 102 applies the analysis mechanism to detect a presence, a progress, and/or a likely onset of one or more types of cancer from evaluation target 132 (e.g., text-based form of patient DNA data). The processing system 102 can generate an evaluation result 134 based on testing the evaluation target 132 with the ML model 104. The processing system 102 can generate the evaluation result 134 that represents a cancer diagnosis or a cancer signal. For example, the evaluation result 134 can represent a determination that the patient has cancer, a stage (e.g., clinically recognized stages 1-4) of the onset cancer, a progress state before/leading up to an onset state of cancer, a likelihood of developing cancer within a predetermined period, an identification of the type of cancer, or a combination thereof.

As an illustrative example, the computing system 100 can include a sourcing device 152 that provides the evaluation target 132 and/or receives the evaluation result 134. The sourcing device 152 can be operated by a patient submitting the evaluation target 132, a healthcare service provider associated with the patient, an insurance company, or the like. Some examples of the sourcing device 152 can include a personal device (e.g., a personal computer, a mobile computing device, such as a smart phone or a tablet, or the like), a workstation, an enterprise device, etc.

As described above, the ML model 104 may be developed and trained based on the DNA information obtained from the unbounded samples. Accordingly, the sourcing device 152 can provide the evaluation target 132 that includes textual representations of DNA information obtained from the unbounded samples. Using the unbounded sample information, the processing system 102 can use or apply the model to generate the evaluation result 134 that provides a signal/score that represents a likelihood that the patient has one or more types of cancer or a measure of proximity to onset of the one or more types of cancer.

In some implementations, the computing system 100 can include a sourcing module 162 operating on the source device 152. The sourcing module 162 can include a device/circuit and/or a software module (e.g., a codec, an app, or the like) that generates or pre-processes the evaluation target 132. For example, the sourcing module 162 can include a homomorphic encoder that encrypts and prevents unauthorized access to the patient data. The evaluation target 132 can include the homomorphically encoded data that can be processed at the processing system 102 without fully decrypting and recovering the patient data. In other words, the processing system 102 can apply the ML model 104 that is configured to process or perform computations on the encrypted data.

The processing system 102 can include a pre-processing module 164 that conditions the evaluation target 132 for and/or during the model application. For example, the pre-processing module 164 can include circuits and/or software instructions that are configured to remove biases or noises introduced before receiving the evaluation target 132 and/or during the processing (e.g., bootstrapping module to remove noise/uncertainties introduced by processing encrypted data) of the evaluation target 132.

### Data Processing Formats

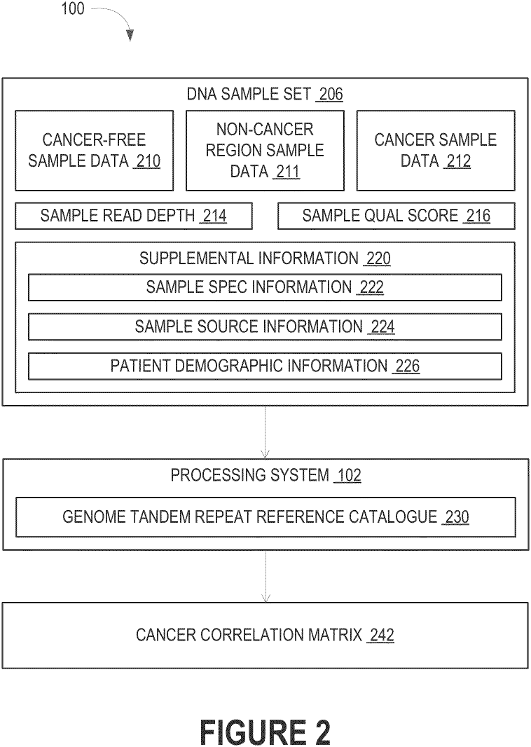

In developing/training the model 104 and/or deploying the model 104, the computing system 100 can utilize a variety of data processing formats (e.g., data structures, organizations, inputs/outputs, or the like). FIG. 2 shows an example data processing formats for the processing system 102 in accordance with one or more implementations of the present technology. The processing system 102 can receive and process a DNA sample set 206 (e.g., an instance of the reference data 112 and/or sample data 130 illustrated in FIG. 1A) having one or more of the formats or subfields illustrated in FIG. 2. Moreover, the processing system 102 can generate the initial feature set 114 (FIG. 1A) using one or more detailed example aspects depicted in FIG. 2.

As an illustrative example, the DNA sample set 206 can include DNA data (e.g., representative of a set of sequenced DNA information) corresponding to different known categories. Examples of the DNA sample set 206 can include genetic information (e.g., text-based representations) derived or extracted from human bodies, such as from tissue extracted during a biopsy or from cell-free DNA (e.g., DNA that is not encapsulated within a cell) in bodily fluids. The DNA sample set 206 can include DNA data collected from volunteers or participating patients having medically confirmed diagnoses and/or from public or private databases.

The DNA sample set 206 can include data collected from different types/categories of samples, such as cancer-free samples (cancer-free data 210), non-cancerous regions/samples (non-regional data 211), and/or cancerous samples (cancer-specific data 212). The cancer-free data 210 can represent text-based DNA data corresponding to samples collected from patients confirmed/diagnosed to be cancer free. The non-regional data 211 can represent text-based DNA data corresponding to the unbounded samples collected from non-cancerous regions (e.g., white blood cells or leukocytes, saliva, or the like) of patients confirmed/diagnosed to have one or more types of cancer. The cancer-specific data 212 can represent text-based DNA data corresponding to samples (e.g., tumor biopsies, liquid biopsies, etc.) collected from cancerous regions or tumors confirmed/diagnosed to be a specified type of cancer. The DNA sample set 206 can include information (e.g., the non-regional data 211 and/or the cancer-specific data 212) corresponding to one or more types of cancers (e.g., breast cancer, lung cancer, colon cancer, and/or the like).

The DNA sample set 206 can further include descriptions regarding a strength or a trustworthiness of the data. For example, the DNA sample set 206 can include a sample read depth 214 and/or a sample quality score 216. The sample read depth 214 can represent a number of times a given nucleotide in the genome (e.g., certain text string/portion) was detected in a sample. The sample read depth 214 may correspond to a sequencing depth associated with processing fragmented sections of the genome within a tissue sample. The sample quality score 216 can represent a quality of identification of the nucleobases generated by DNA sequencing. In some implementations, the sample quality score 216 can include a phred quality score.

The DNA sample set 206 can also include supplemental information 220 that describes other aspects of the sample or the source of the data. For example, the supplemental information 220 can include information such as sample specification information 222 (or simply “specification information”), sample source information 224 (or simply “source information”), patient demographic information 226, or a combination thereof.

The specification information 222 can include technical information or specifications about the sequenced DNA associated with the DNA sample set 206. For example, the specification information 222 can include information about the locations 118 (FIG. 1A) within the genome to which the DNA fragments (e.g., portions of DNA) correspond, such as intron and exon regions, specific genes, or chromosomes. Also, the specification information 222 can describe, e.g., (1) the process, methods, and instrumentation used to extract and sequence the genetic material, (2) the number of sequencing reads for each sample, or a combination thereof.

The source information 224 can include details regarding the source and/or the categorization of the sample. For example, the source information 224 can include information about the cancer type, the stage of cancer development, the organ or tissue from which the sample was extracted, or a combination thereof.

The patient demographic information 226 can include demographic details of the patient from which the sample was taken. For example, the patient demographic information 226 can include the age, the gender, the ethnicity, the geographic location of where the patient resides/visited, the duration of residence/visitation, predispositions for genetic disorders or cancer development, family history, or a combination thereof.

The processing system 102 can analyze the DNA sample set 206 using the mutation analysis mechanism. Accordingly, the processing system 102 can identify mutations or mutation patterns in specific DNA sequences that can be used as markers to determine the existence, the progress, and/or the developing stages of a particular form of cancer. To identify the relevant mutations, the processing system 102 can detect a set of targeted locations or text patterns (according to, e.g., the TRs) within the reference genomes.

The processing system 102 can generate and/or utilize a genome tandem repeat reference catalogue 230 that represents a catalogue or a collection of uniquely identifiable TRs in the human genome. The genome tandem repeat reference catalogue 230 can include the unique segment set 113 of FIG. 1A. As an example, the genome tandem repeat reference catalogue 230 can be based on a reference human genome (e.g., the reference data 112), such as the GRCh38 reference genome. The uniquely identifiable sequences can include DNA sequences having therein a series of multiple instances of directly adjacent identical repeating nucleotide units or base patterns, such as microsatellite DNA sequences. The base patterns can have a predetermined length, such as one for a repetition of one letter or monomer (e.g., ‘AAAA’) or greater (e.g., four for tetramers, such as ‘ACTG’). Such uniquely identifiable TRs can serve as reference sequences (e.g., reference locations within the human genome) or markers for evaluating the DNA sample set 206. Since the DNA sample set 206 may correspond to incomplete portions of DNA, the unique TRs found within the fragments may be used to map the DNA information to the human genome.

The processing system 102 can use the genome tandem repeat reference catalogue 230 to compute the initial feature set 114. For example, the processing system 102 can use the unique TRs identified in the genome tandem repeat reference catalogue 230 to generate derived strings that represent potential mutations. In some implementations, the processing system 102 can identify text characters preceding and/or following each unique TR and derive the mutation strings that represent one or more types of mutations (e.g., insert-deletion (indel) mutations). Details regarding the initial feature set 114 (e.g., strings with flanking characters and/or mutation strings) are described below.

The processing system 102 can compare the mutations at the targeted locations/patterns across the different types of DNA sample set 206. Based on the comparison, the processing system 102 can compute a correlation between or a likely contribution of the mutations at the targeted locations/sequences and the development of cancer. Accordingly, the processing system 102 may generate a cancer correlation matrix 242 that correlates identified tumorous sequences or text-based patterns to specific types of cancer. For example, the cancer correlation matrix 242 can be an index that includes multiple instances of the uniquely identifiable tandem repeat sequences in the genome TR reference catalogue 230 that, when found to be tumorous, indicate the existence of a particular form of cancer or indicate the possibility that a particular form of cancer will develop.

The processing system 102 can perform the feature selection using the cancer correlation matrix 242, such as by retaining the locations/patterns and/or derived mutation patterns having at least a predetermined degree of correlation to one or more corresponding types of cancer. Using the selected features, the processing system 102 can develop and train the ML model 104 configured to detect, predict, and/or evaluate development or onset of cancer.

### Base Text Patterns - Expected Phrases

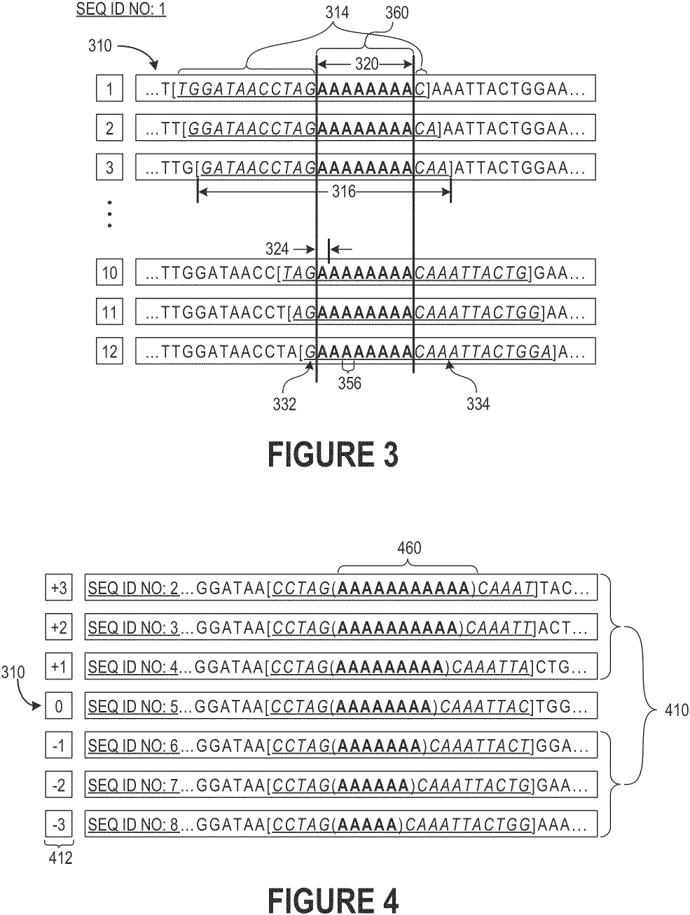

The processing system 102 can use segments (e.g., the unique segment set 113) to generate phrases. FIG. 3 shows example expected phrases 310 in accordance with one or more implementations of the present technology. The expected phrases 310 can correspond to textual representations of the DNA sequences or a set of sequence variations that may be used as bases for subsequent processing/comparisons, such as in deriving mutations strings and analyzing the DNA sample set 206 (FIG. 2).

For context, samples collected from patients may include fragments or portions of the overall DNA. As such, the corresponding sequenced values or the text string may include different combinations of characters. The processing system 102 (FIG. 1A) can generate the expected phrases 310 as representations of different character combinations that include the uniquely identifiable segments (e.g., the unique segment set 113). In some implementations, the processing system 102 can generate a set (illustrated as a unique sequence identifier number in FIG. 3) of the expected phrases 310 for each unique segment 360 (illustrated using bolded characters in FIG. 3).

The expected phrases 310 can have a phrase length 316 of k (e.g., between 10 to 50 or more) number of DNA base pairs or pairs of nucleobases. Each DNA base pair can be represented as a single text character (e.g., ‘A’ for adenine, ‘C’ for cytosine, ‘G’ guanine, and ‘T’ thymine). As such, the expected phrases 310 may also be referred to as “k-mers.”

In some implementations, as described above, the unique segment 360 can include a DNA sequence, of a specified minimum length. The unique segment 360 can include a series of multiple instances of directly adjacent identical repeating nucleotide units or repeated base units 356. For example, the unique segment 360 can include a minisatellite DNA or microsatellite DNA sequence of a specified minimum length. Accordingly, the unique segment 360 can correspond to a repeated pattern of the repeated base units 356, and the number of repetitions can correspond to a segment length 320 (e.g., the total length of, or total number of, nucleotide base pairs) for the unique segment 360. The repeated base unit 356 can have a base unit length 324 corresponding to the number of nucleotides within the repeated base unit 356 (e.g., one for a mono-nucleotide, two for a di-nucleotide, etc.).

For illustrative purposes, FIG. 3 shows a specific instance for the unique segment 360 of “AAAAAAAA,” annotated as “A8,” located at the molecular position starting at “10,513,372” on chromosome 22. In this example, the unique segment 360 includes the segment length 320 of eight base pairs with the repeated base unit 356 of one base pair (e.g., a monomer or a mono-nucleotide) ‘A.’

The processing system 102 can use the phrase length 316 (e.g., k between 10 to 50 or more base pairs) that has been predetermined or selected to capture targeted amount of data/characters surrounding the unique segments 360. As such, the phrase length 316 can be greater than the segment length 320, and each of the expected phrases 310 can include a set of flanking texts 314 (e.g., text-based patterns, illustrated using italics in FIG. 3) preceding and/or following the corresponding unique segment 360.

The processing system 102 can generate the expected phrases 310 in a variety of ways. As an illustrative example, the processing system 102 can use each of the unique segments 360 as an anchor for a sliding window having a length matching the phrase length 316. The processing system 102 can iteratively move the sliding window relative to the unique segment 360 and log the text captured within the window as an instance of the expected phrases 310. As such, each of the expected phrases 310 can correspond to a unique position of the sliding window relative to the unique segment 360. Also, the set of expected phrases 310 for one reference TR can include different combinations of the flanking text 314 (e.g., a combination of one or more leading characters 332 and/or one or more tailing characters 334.

The total number of base pairs in flanking text 314 can be a fixed value that is based on the phrase length 316 and the segment length 320. The number of characters in the flanking text can be calculated as the difference between the phrase length 316 and the segment length 320. As an example, for one of phrases having a length of 21 base pairs and a segment length of 8 base pairs, the flanking text can include13 base pairs/characters.

Each of the expected phrases 310 can represent one of a number of position variant k-mers based on the flanking texts 314. The position variant k-mers can include specific numbers of base pairs in the expected flanking text 332 and tailing flanking text 334. For example, a set of the expected phrases 310 can include the same unique segment (e.g., repeated pattern of the TR) and differ from one another according to the number of base pairs included in the leading flanking text 332 and/or the tailing flanking text 334. In general, the number of base pairs included in the leading flanking text 332 and tailing flanking text 334 can vary inversely between the different instances of the position variant k-mers or expected phrases 310.

As an example, each of the expected phrases 310 illustrated in FIG. 3 has the phrase length 316 of 21 base pairs and the segment length 320 of 8 base pairs. A first expected phrase can have the leading characters 332 corresponding to 12 base pairs and the tailing character 334 corresponding to 1 base pair. A second expected phrase can have the leading characters 332 corresponding to 11 base pairs and the tailing characters 334 of 2 base pairs. The pattern can be repeated until the last expected phrase has the leading characters 332 corresponding to 1 base pair and the tailing characters 334 corresponding to 12 base pairs.

The expected phrases 310 can be grouped into sets that each correspond to a unique segment as described above. The total number of phrases or position variant k-mers (position variant total) in the grouped set can be represented as:

$$\text{Position Variant Total =} \left(\text{Phrase length} k\right) \text{-} \left(\text{Segment length}\right) \text{- 1} \text{.}$$

For the example illustrated in FIG. 3, the set of expected phrases can have a position variant total of 12, representing 12 different instances of phrases corresponding to the phrase length 316 of 21 and the segment length 320 of 8.

In some implementations, the processing system 102 can use the unique instances of the TRs as the basis for generating the sets of expected phrases 310. Accordingly, each of the expected phrases 310 can also be unique since it is generated using the corresponding unique TR as a basis. The processing system 102 can use the unique expected phrases 310 to account for and identify the fragmentations likely to be included in the patient samples.

### Base Text Patterns - Derived Phrases

The processing system 102 can use the expected phrases to analyzes mutations in genetic information (e.g., sequenced DNA segments), such as for detecting tumorous/cancerous DNA sequences. The expected phrases can be used to detect locations within the reference genome and related mutations that are indicative of certain types of cancers or likely onset thereof. The processing system 102 can use the expected phrases as basis to generate derived phrases that represent various mutations in the genetic information. The processing system 102 can use the derived phrases to recognize or detect mutations in the DNA sample set 206 (FIG. 2), the sample data 130 (FIG. 1A), or the like in developing, training, and/or deploying the ML model 104. Effectively, the processing system 102 can identify the mutation patterns indicative of certain types of cancers based on using the derived phrases to determine differences between healthy and cancerous DNA samples (between, e.g., the cancer-free data 210, the non-regional data 211, and/or the cancer-specific data 212 illustrated in FIG. 2).

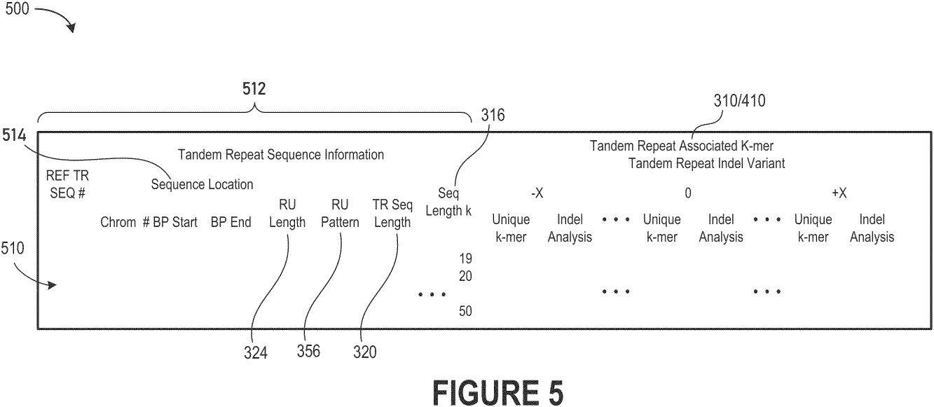

FIG. 4 shows example derived phrases 410 in accordance with one or more implementations of the present technology. The processing system 102 (FIG. 1A) can generate the derived phrases 410 based on adjusting the expected phrases 310 expected to a predetermined pattern. For example, for one or more or each expected phrase 310, the processing system 102 can generate a set of the derived phrases 410 that represent indel mutations of the corresponding expected phrase 310. In some implementations, the processing system 102 can generate the set of derived phrases 410 that correspond to a predetermined number of insertions and/or deletions in the unique segment 360 (FIG. 3) within the corresponding expected phrase 310. In other words, the set of derived phrases 410 can represent the indel variants of the sequence represented by the corresponding expected phrase 310.

The processing system 102 can generate the set of the derived phrases 410 based on adjusting (via insertion/deletion) the number of the repeated base units 356 (FIG. 3) and/or one or more characters in the unique segment 360 of the expected phrase 310. Accordingly, the processing system 102 can generate a set of derived segments 460 that correspond to indel variants of the unique segment 360.

The processing system 102 can generate the derived phrases 410 based on adding and/or adjusting the flanking text 314 (FIG. 3) around the derived segments 460 (illustrated as the bolded characters within parentheses ‘()’). In some implementations, the processing system 102 can generate the derived phrases 410 having the same phrase length 316 (FIG. 3) as the expected phrases 310. As a result, the processing system 102 can expand or reduce the coverage of the flanking text 314 according to the indel changes to the unique segment 360 (e.g., the originating pattern of TRs). With deletions, the processing system 102 can include corresponding number of new characters from the overall sequence into the flanking text 314 (FIG. 3). Similarly with additions, the processing system 102 can remove the corresponding number of characters from the flanking text 314. For illustrative purposes, FIG. 4 shows the surrounding adjustments occurring in the trailing characters 334 (FIG. 3) while maintaining the leading characters 332 (FIG. 3). However, it is understood that the processing system 102 can operate differently, such as by (1) adjusting the leading characters 332 while maintaining the trailing characters 334 and/or (2) spreading the adjustments across the leading characters 332 and the trailing characters 334 according to the number of characters in the original phrase and/or a predetermined pattern.

For the example illustrated in FIG. 4, the expected phrase 310 can correspond to the repeated TR segment of “AAAAAAAA” or A8 beginning at position 10,513,372 on chromosome 22. The derived phrases 410 can correspond to the derived segments 460 including up to three insertions and deletions of the repeated base unit ‘A.’ In other words, the derived phrases 410 can correspond to phrases built around A5, A6, A7, A9, A10, and A11.

The number of the derived phrases 410 associated with a given expected phrase can be determined by an indel variant value 412. The indel variant value 412 can include an integer value representative of the number of insertions and deletions. The indel variant value 412 can further function as an identifier for a phrase. For example, the indel variant value ‘0’ can represent the expected phrase 310 having zero insertions/deletions. Positive indel variant values (e.g., 1, 2, 3) can represent derived phrases including corresponding number of insertions of base units or characters in the repeated TR portion. Negative indel variant values (e.g., -1, -2, -3) can represent derived phrases corresponding number of deletions of base units or characters in the repeated TR portion. For the example illustrated in FIG. 4, the indel variant values 1, 2, and 3 can represent/identify A9, A10, and A11, respectively. Also, the indel variant values -1, -2, and -3 can represent A7, A6, and A5, respectively.

For context, the processing system 102 can use the expected phrases 310 and the corresponding sets of derived phrases 410 to analyze the DNA sample set 206 and develop/test the ML model 104 (FIG. 1A). The phrases generated using the unique TR patterns can provide accurate and precise identification of corresponding sequences in the different types of health and cancerous DNA samples. In other words, the various phrases can represent the type of textual patterns or the corresponding sequences that are targeted for analyses and comparisons between the cancer-free data 210, the non-regional data 211, and/or the cancer-specific data 212. For example, the processing system 102 can use the various phrases to identify the numbers and types/locations of mutations in the cancer-related samples and absent in healthy samples. The processing system 102 can aggregate the results across multiple samples and patients to derive a pattern or a correlation between certain types of mutations and the onset of certain types of cancer.

To put things another way, the processing system 102 can identify unique patterns (e.g., the unique TR patterns and/or the corresponding expected phrases 310) that each occur once within the human genome. The unique patterns can be used to identify specific locations and portions within the human genome for various analyses. Moreover, the processing system 102 can target specific types of mutations, such as indel mutations, in developing a cancer-screening and/or a cancer-predicting tool. It has been found that various types of cancers can be accurately detected and progress/status of such types of cancers can be described using the expected phrases 310 and the corresponding sets of the derived phrases 410 (e.g., sequences identified using unique TR-based patterns and indel variants thereof) and without considering other aspects/mutations of the human DNA. As a result, the processing system 102 can generate the ML model 104 that can accurately detect the existence, predict a likely onset, and/or describe a progress of certain types of cancers using the various phrases. In other words, the processing system 102 can detect/predict the onset of cancer without processing the entire DNA sequence and different types of mutation patterns.

The processing system 102 can further improve the efficiency and reduce the resource consumption using the indel variant value 412. Given the downstream processing methodology, the indel variant value 412 can control the number of phrases considered in developing/training the ML model 104 and thereby affect the overall number of computations and the amount of resource consumption. When the indel variant value 412 is too high, the processing system 102 may end up analyzing a reduced or ineffective number of possible sequences. For example, as the total number of base pairs in the TR indel variant approaches the phrase length 316, the number of available derived phrases and the likely occurrence of such mutations decrease. Accordingly, in some implementations, the indel variant value 412 in the range of three to five provides sufficient coverage for varying degrees of possible insertion and deletion mutations that are indicative of one or more types of cancer. This range of values may be sufficient to provide accurate results without requiring ineffective or inefficient amount of computing resources.

Additionally, the processing system 102 can further improve the efficiency and reduce the resource consumption using the segment length 320 (e.g., the length of the uniquely identifiable TR-based pattern). It has been found that the probability of mutation occurrences decreases as the tandem repeat segment length 320 is reduced. In particular, the mutation rate for genome TR sequences with segment length 320 of fewer than five base pairs is significantly less than genome TR sequences with the segment length 320 of five or more base pairs. Thus, the expected phrases 310 can be selected as the genome TR sequence with the segment length 320 of five or greater.

### Base Text Patterns - Storage/Tracking

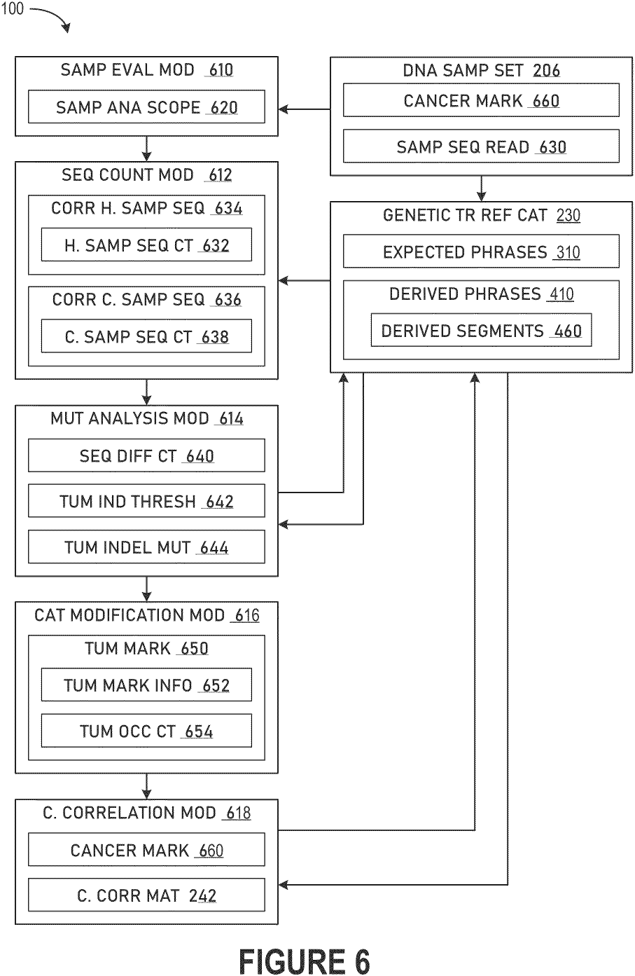

The processing system 102 can store the various phrases (e.g., the expected phrases 310 and/or the corresponding sets of the derived phrases 410) in the genome TR reference catalogue 230 (FIG. 2). FIG. 5 shows an example analysis template 500 in accordance with one or more implementations of the present technology. The processing system 102 can use the analysis template 500 to represent the various phrases and/or track the associated processing results.

In some implementations, the analysis template 500 can correspond to a format for the genome TR reference catalogue 230. The genome TR reference catalogue 230 can include catalogue entries 510 for each instance of the unique segments 360 (e.g., uniquely identifiable or reference TR patterns) or a unique combination/set of segments. The entries 510 can include TR sequence information 512 that characterizes the unique segments 360 and/or the derived segments 460. For example, the TR sequence information 512 can include a sequence location 514, the segment length 320, the base unit length 324, the repeated base unit 356, a position representative of combined (e.g., mathematically combined, such as according to a predetermined formula), or a combination thereof.

The sequence location 514 can identify the location of the corresponding unique segment 360 and/or expected phrase 310 within the reference genome. As an example, the sequence location 514 can be described based on the molecular location of the unique segment 360, such as (1) the chromosome on which the TR sequence is located and/or (2) the base pair numbers in the chromosome marking the beginning/end of the TR sequence. The sequence location 514 can act as a unique identifier that distinguishes one instance of the unique segment 360 and/or the expected phrase 310 from another. For example, the expected phrases 310 that share the same repeated base unit 356 and the base unit length 324 can be distinguished from one another based on the sequence location 514.

The entries 510 for each instance of the unique segment 360 can include information for one or more instances of the corresponding phrases (e.g., expected and/or derived). For example, the entries 510 can include information for the expected phrases 310 and/or the derived phrases 410 with various values for the phrase length 316. For illustrative purposes, this instance of entries 510 is shown including information for the expected phrases 310 with phrase lengths corresponding from 19 base pairs to 60 base pairs. However, it is understood that the entries 510 can include information regarding fewer than 19 base pairs and/or more than 60 base pairs. As another example, the entries 510 can include information that distinguishes between the expected phrases 310 and the derived phrases 410. In some implementations, the entries 510 can identify the expected phrases 310 associated with a corresponding TR pattern. For instance, the TR pattern A8 beginning at position 10,513,372 can yield 16 sequences or expected phrases 310 having the phrase length 316 of 30 base pairs.

The entries 510 can further identify the derived phrases 410 that are absent from the reference genome. For illustrative purposes, Table 1 below summarizes the derived phrases 410 having the segment length 316 of 30 base pairs for the unique segment 360 or TR pattern of “A8” beginning at position 10,513,372 (annotated as ‘372) on chromosome 22. In this example, each of the derived phrases 410 corresponding to indel variants with the indel variant value 412 ranging from “-5” to “+5” are not found in the reference genome.

TABLE 1

Chromosome 22, ‘372, “A8” Reference TR Associated Indel Phrase Summary

| Indel Variant Value | Position Variant Total | Total That Do Not Appear |
|---|---|---|
| +5 | 16 | 16 |
| +4 | 17 | 17 |
| +3 | 18 | 18 |
| +2 | 19 | 19 |
| +1 | 20 | 20 |
| -1 | 22 | 22 |
| -2 | 23 | 23 |
| -3 | 24 | 24 |
| -4 | 25 | 25 |
| -5 | 26 | 26 |

The analysis template 500 can be used to track the statistical data generated during development/training of the ML model 104. For example, the processing system 102 can track the occurrences of certain mutations according to the sequence location 514 or the identifier for the corresponding entry 510 and the indel mutation offset/identifier. The processing system 102 can use the counted occurrences for each sample, each sample set, or a combination thereof to compute the correlation between the mutations and the onset of the corresponding type of cancer.

The analysis template 500 is shown for exemplary purposes as a template with a general layout for organizing information for each of the segments and/or phrases. It is understood that the analysis template 500 can include different categorizations and arrangements with additional or different pieces of information. Further, it is understood that an active or “in use” version of the genome TR reference catalogue 230 can be populated with values corresponding to the various categories of the entries 510.

### Control Flow

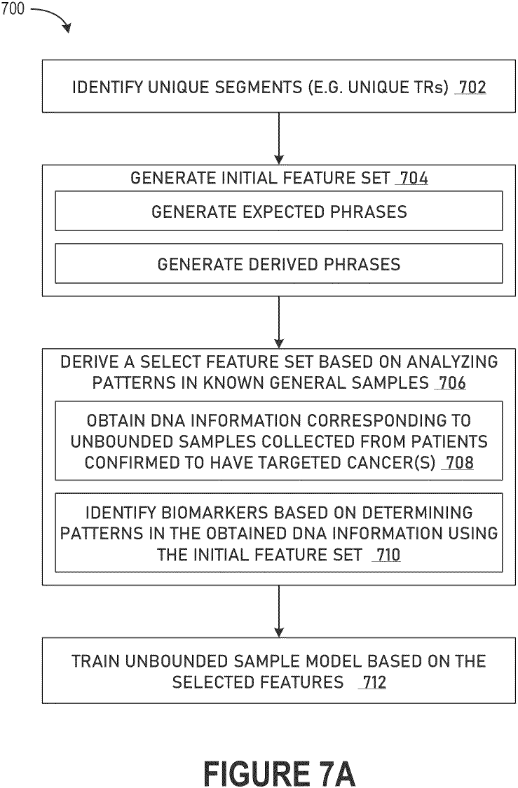

FIG. 6 shows a control flow diagram illustrating the functions of the computing system 100 in accordance with one or more implementations of the present technology. The computing system 100 can be implemented to supplement and refine information in the genome TR reference catalogue 230 with information from the DNA sample sets 206 based on the unique segments 360 and the various phrases. In general, the computing system 100 can analyze one or more of the DNA sample sets 206 to process (1) mutations at specific locations of DNA sequences, (2) correlation of mutation patterns, (3) corresponding indications of one or more types of cancer, or a combination thereof. The functions of the computing system 100 can be implemented with a sample set evaluation module 610, a sequence count module 612, a mutation analysis module 614, a catalogue modification module 616, a cancer correlation module 618, or a combination thereof.

The evaluation module 610 can be configured evaluate the scope of the DNA sample set 206, including the cancer-free data 210, the non-regional data 211, and/or the cancer-specific data 212. For example, the evaluation module 610 can evaluate the DNA sample set 206 to identify factors, properties, or characteristics thereof to facilitate analysis of the different categories of data. In some implementations, the evaluation module 610 can be optional. The evaluation module 610 can generate a sample analysis scope 620 for the DNA sample set 206. The sample analysis scope 620 is a set of one or more factors that may govern/control the analysis of the DNA sample set 206. For example, the sample analysis scope 620 can be generated based on the supplemental information 220. The sample analysis scope 620 can be used to identify usable phrases (e.g., the expected phrases 310 and/or the derived phrases 410) based on the sequence location 514 and the phrase length k 316.

The computing system 100 can receive the derived phrases 410 and associated information from the genome TR reference catalogue 230 and/or the DNA sample set 206. The mutation analysis mechanism can be implemented with the count module 612 and the analysis module 614. The count module 612 may be responsible for calculating a number of occurrences (e.g., a sequence count) for specific DNA sequences/phrase in a sample set. The count module 612 can calculate the sequence count based on a number of sample sequence reads 630, such as the sequence reads for the portions of DNA in one or more categories of data in the DNA sample set 206.

For the cancer-free data 210, the count module 612 can calculate a healthy sample sequence count 632 for each instance of a corresponding healthy sample sequence 634 identified in the cancer-free data 210. The corresponding healthy sample sequence 634 is a DNA sequence in the healthy sample DNA information 634 that corresponds to one of the derived segments 460 and/or the derived phrases 410. The heathy sample sequence count 632 is the number of times that the corresponding healthy sample sequence 634 is identified in the cancer-free data 210. Similarly, for the cancer-specific data 212 and/or the non-regional data 211, the count module 612 can calculate count values for each instance of a targeted sequence identified in the data group. In other words, the count module 612 can calculate the number of times the various phrases are found within the samples according to the corresponding groups/categories.

The count module 612 can identify the corresponding healthy sample sequence 634 and the corresponding cancerous sample sequence 638 for a given expected phrase, and more specifically the derived phrase. For example, the sequence count module 612 can search through the different categories of data for matches to one or more of the derived segments within the corresponding phrases. As one specific example, the count module 612 can search for a string of consecutive base pairs that matches one of the derived segments 460 of the derived phrases 410.

The count module 612 can calculate the healthy sample sequence count 632 as the total number of each of the corresponding healthy sample sequence 634 identified in each of the sample sequence reads 630 in the cancer-free data 210. In many cases, the corresponding healthy sample sequence 634 will correspond with a single instance of the tandem repeat indel variants. In these cases, the total value of the healthy sample sequence count 632 will be equal to the total number of the sample sequence reads 630 in the cancer-free data 210. For example, where the cancer-free data 210 includes 50 instances of the sample sequence reads 630 per DNA segment, the healthy sample sequence count 632 for a given instance of the corresponding healthy sample sequence 634 should also be 50. The case of non-unity between the number of sequencing reads and the healthy sample sequence count 632 can generally be attributed to sequencing errors.

In many cases, the corresponding healthy sample sequence 634 will match with the phrase with the indel variant value 312 of zero (e.g., the expected phrase with no insertions or deletions of the unique segment 360). However, in some cases, the corresponding healthy sample sequence 634 can differ. The differences between the corresponding healthy sample sequence 634 and the phrase with the indel variant value 312 of zero can account for wild type variants (e.g., naturally occurring variations) in the cancer-free data 210.

Similarly, the count module 612 can calculate the cancerous sample sequence count 636 for each of the corresponding cancerous sample sequence 638 that appear in the sample sequence reads 630 in the cancer-specific data 212. Due to possible mutations, the cancer-specific data 212 can include multiple different instances of the corresponding cancerous sample sequence 638 matching different instances of the derived segments 460, with each corresponding cancerous sample sequence 638 having varying values of the cancerous sample sequence count 636. As an example, in some cases, the corresponding cancerous sample sequence 638 and cancerous sample sequence count 636 will match with the corresponding heathy sample sequence count 634 and healthy sample sequence count 632, indicating no mutations. As another example, for a given instance of the derived phrase 410, the cancer-specific data 212 may have a split in the cancerous sample sequence count 636 between the cancerous sample sequence 638 that is the same as the corresponding healthy sample sequence 634 and one or more other instances of the tandem repeat indel variants. For a given instance of the derived phrase 410, the count module 612 can track the cancerous sample sequence count 636 for each different instance of the corresponding cancerous sample sequence 638 in the cancer-specific data 212.

The flow can continue to the analysis module 614. The analysis module 614 may be responsible for determining whether a mutation exists in the corresponding cancerous sample sequence 638 of the cancer-specific data 212. In general, the existence of a mutation in the cancer-specific data 212 can be determined based on differences in the repeated TR patterns between the corresponding heathy sample sequence 634 and the corresponding cancerous sample sequence 638. More specifically, a difference in the number of the repeated base unit 356 can represent the existence of an indel mutation (e.g., a mutation corresponding to an insertion or a deletion of the repeated TR unit), such as for cancer-specific data 212 in comparison to the cancer-free data 210. For example, the analysis module 614 can determine that a mutation exists when the corresponding cancerous sample sequence 638 matches one of the derived segments 460 and/or the derived phrases different from that of the corresponding healthy sample sequence 634. In another example, the analysis module 614 can determine the difference between the corresponding healthy sample sequence 634 and the corresponding cancerous sample sequence 638 based on a sequence different count 640 (e.g., the total number of corresponding cancerous sample sequences 638 differing from the corresponding healthy sample sequences 634). In the case where the sequence difference count 640 indicates no differences, such as when the sequence difference count 640 is zero, the analysis module 614 can determine that no mutation exists in the corresponding cancerous sample sequence 638.

In general, the analysis module 614 can determine that an indel mutation has occurred when the sequence difference count 640 is a non-zero value. In some implementations, the analysis module 614 determines whether the indel mutation is a tumorous indel mutation based on whether the sequence difference count 640 is greater than the error percentage of the approach or apparatus used to sequence the cancer-free data 210, cancer-specific data 212, or a combination thereof.

In another implementation, the analysis module 614 can determine whether the indel mutation is a tumorous indel mutation 644 based on a tumor indication threshold 642. The tumor indication threshold 642 is an indicator of whether the number of mutations for a particular sequence in the cancer-specific data 212 indicates the existence of a tumorous indel mutation 644. The tumorous indel mutation 644 may occur when the sequence difference count 640 exceeds a tumor indication threshold 642. As an example, the tumor indication threshold 642 can be based on a percentage between the total number of sample sequence reads 630 and the sequence difference count 640. As a specific example, the tumor indication threshold 642 can require a sequence different count 640 to be greater than 70 percent of the sample sequence reads 630 for the cancer-specific data 212. In another specific example, the tumor indication threshold 642 can require the sequence difference count 640 to be greater than 80 percent of the sample sequence reads 630 for the cancer-specific data 212. In another specific example, the tumor indication threshold 642 require the sequence difference count 640 to be greater than 90 percent of the sample sequence reads 630 for the cancer-specific data 212.

When the corresponding cancerous sample sequence 638 includes the tumorous indel mutation 644, the computing system 100 can implement the modification module 616 to update or modify the genome TR reference catalogue 230. Said another way, the computing system 100 can implement the modification module 616 responsive to determining that the corresponding cancerous sample sequence 638 includes the tumorous indel mutation 644. For example, the modification module 616 can modify the genome TR reference catalogue 230 by identifying the instance of the catalogue entries 510 as a tumor marker 650 when the tumorous indel mutation 644 exists in the corresponding cancerous sample sequence 638.

The catalogue entries 510 that are identified as a tumor marker 650 can be modified by the modification module 616 to include tumor marker information 652. Some examples of the tumor marker information 652 can include a tumor occurrence count 654, such as the number of times that the tumorous indel mutation 644 was identified in a particular instance of the segment/phrase (e.g., TR pattern) for a given form of cancer. As a specific example, the tumor occurrence count 654 can be compiled from analysis for the DNA sample sets 206 for numerous cancer patients.

In another example, the tumor marker identification 652 can include information about the different instances of the corresponding cancerous sample sequence 638 matching to different instances of the derived segments/phrases along with the cancerous sample sequence count 636, the total number of sample sequence reads 630 of the DNA sample set 206, all or portions of the supplemental information 220, or a combination thereof. In a further example, the tumor marker information 652 can include the number of repeated base units 356 in the corresponding cancerous sample sequence 638 that were different from the corresponding healthy sample sequence 634.

The tumor marker information 652 can include information based on the supplemental information 220. For example, the tumor marker information 652 can include the supplemental information 220 (e.g., source information), such as the cancer type, the stage of cancer development, organ or tissue from which the sample was extracted, or a combination thereof. In another example, the tumor marker information 652 can include the supplemental information 220 of the patient demographic information, such as the age, the gender, the ethnicity, the geographic location of where the patient resides or has been, the duration of time that the patient stayed or resided at the geographic location, predispositions for genetic disorders or cancer development, or a combination thereof.

The computing system 100 can use one or more instances of the segments/phrases identified as the tumor marker 650 to generate the cancer correlation matrix 242 with the correlation module 618. For example, the correlation module 618 can identify cancer markers 660 based on the tumor occurrence count 654 for each of the tumor markers 650 in the genome TR reference catalogue 230. The cancer markers 660 can correspond to mutation hotspots that are specific to indel mutations in instances of the TR patterns. In one implementation, the correlation module 618 can identify the cancer markers 660 based on regression analysis. For example, the regression analysis can be performed with a receiver operating characteristic curve to the optimum sensitivity and specificity from the tumor markers 650, tumor occurrence count 654, or a combination thereof to determine the cancer markers 660.

In another implementation, the correlation module 618 can identify the cancer markers 660 based on a ratio between, or percentage of, the tumor occurrence count 654 for the tumor marker 650 and the total number of the DNA sample sets 206 of a particular form of cancer that have been analyzed for the tumor marker 650. As a specific example, the correlation module 618 can identify the cancer markers 660 as the tumor markers 650 when the ratio between the tumor occurrence count 654 and the total number of DNA sample sets 206 that are analyzed is 90 percent or more of the DNA sample sets 206 for a particular form of cancer. In this case, the cancer correlation matrix 242 can include the cancer markers 660 that were identified in this manner.

In a further implementation, the correlation module 618 generates the cancer correlation matrix 242 as the tumor markers 650 that are common among a percentage of the DNA sample sets 206 for a particular form of cancer are found. For example, the correlation module 618 can generate the cancer correlation matrix 242 as the tumor markers 650 appear in 90 percent or more of the total number of DNA sample sets 206. In other implementations, the correlation module 618 can generate the cancer correlation matrix 242 through other methods, such as regression analysis or clustering.

The correlation module 618 can generate the cancer correlation matrix 242 taking into account the supplemental information 220, such as the patient demographic information, to generate the cancer correlation matrix 242 for subpopulations. For example, the correlation module 618 can generate the cancer correlation matrix 242 based on the patient demographic information specific to gender, nationality, geographic location, occupation, age, another characteristic, or a combination of characteristics.

The computing system 100 has been described in the context of modules that perform, serve, or support certain functions as an example. The computing system 100 can partition or order the modules differently. For example, the evaluation module 610 could be implemented on the processing system 102, while the count module 612, analysis module 614, and correlation module 618 could be implemented on an external device. Alternatively, the processing system 102 can include the various modules described above.

### Approaches to Developing and Training the Model

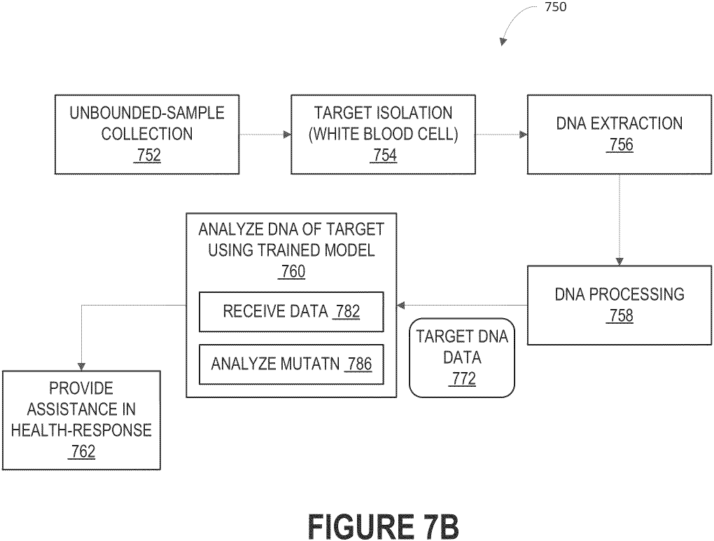

As described above, the system 100 can develop and generate the ML model 104 of FIG. 1A using the processing system 102 of FIG. 1A and/or one or more modules described above. FIG. 7A show a flows chart of an example method 700 of operating the system 100 to develop and/or train the ML model 104 in accordance with one or more implementations of the present technology. The method 700 can be implemented by the processing system 102 and or one or more modules illustrated in FIG. 6.

At block 702, the system 100 can identify unique segments (e.g., the unique segment set 113 of FIG. 1A). For example, the system 100 can identify the set of unique TRs in the reference data 112 of FIG. 1A (e.g., the human genome). The unique segment set 113 can represent unique portions within the human genome. In some implementations, the system 100 can access the reference data 112 that was predetermined and stored at an accessible location.

At block 704, the system 100 can generate an initial feature set (e.g., the initial feature set 114 of FIG. 1A) using the identified unique segments. For example, the system 100 can identify the set of expected phrases that include the unique segments. As described above, each expected phrase can include a unique combination of flanking text before, after, or both relative to the unique segment (e.g., unique TRs).

Additionally or alternatively, the system 100 can compute derivations (e.g., representations of mutations, such as indel mutations) of the expected phrases as described above. For example, the system 100 can generate the set of derived phrases that each represent a unique somatic indel variant/mutation of the expected phrase.

At block 706, the system 100 can derive a select set of features or phrases (e.g., the set of features 124 of FIG. 1A). The system 100 can derive the set of features 124 based on analyzing and detecting patterns in samples known to have been collected from patients having cancer (e.g., the unbounded samples, such as the non-regional data 211 of FIG. 2). The system 100 can use the initial feature set to analyze and detect the patterns.

As an illustrative example, at block 708, the system 100 can obtain DNA information corresponding to unbounded samples (e.g., leukocyte, saliva, cheek swabs, or the like) collected form patients confirmed to have one or more targeted types of cancers. The collection locations for the unbounded samples can be used to compute cancer signals corresponding to unrelated locations, such as for lung cancer, brain cancer, breast cancer, etc. Moreover, as described above, the selected features can be derived based on analyzing the unbounded samples directly for indications of the type(s) of cancer instead of using the unbounded samples as control for analyzing other cancerous samples. In some implementations, the system 100 can obtain the DNA information from databases or repositories that provide the unbounded samples (e.g., the leukocyte data) for different purposes, such as to be used as control data for other types of analysis.

At block 710, the system 100 can use the obtained DNA information of unbounded samples to identify the biomarkers (e.g., the select features) therein. In other words, the system 100 can derive the select features within the initial feature set 114 that have at least a threshold amount of influence or correspondence to the associated type of cancer. For example, the system 100 can identify the text sequences that represent the mutations, found in the unbounded samples, that are characteristic or indicative of the corresponding cancer.

It has been discovered that the identified biomarkers are different from the DNA-based biomarkers found within the cancerous locations or tumors. The biomarkers associated with the cancerous locations or tumors can represent the causes for the corresponding type of tumor. In contrast, the biomarkers for the unbounded samples can represent mutations that are related to or caused by the unbounded samples interacting with the cause of the cancer. For example, the biomarkers in the leukocytes can represent the changes therein caused by their physiological interactions with the tumor cells.

The unique segment set 113, the initial feature set 114, or a combination thereof provide capability for the system 100 to identify the biomarkers in the unbounded samples that indicate the existence or the proximity to the onset of one or more cancers. The unique segment set 113, the initial feature set 114, or a combination thereof can provide discrete text strings that drastically reduce the required processing resources in comparison to the overall human genome and the full set of potential mutations. As such, the unique segment set 113, the initial feature set 114, or a combination thereof allow the system 100 to practically analyze the DNA information and identify the biomarkers, even in unbounded samples.

At block 712, the system 100 can use the selected features to develop and train the corresponding ML model 104 (e.g., the unbounded sample model). The system 100 can develop the ML model 104 according to one or more ML mechanisms, such as neural network, random forest, support vector machine (SVM), or the like. The ML model 104 can be configured to compute a cancer signal that represents (1) a likelihood that a corresponding patient has developed one or more types of cancer or (2) a development status at least leading up to or recovering from the onset of the one or more types of cancer.

The system 100 can train the ML model with a set of training data including the text strings representative of DNA information of other/separate patients’ samples. The training data can include the cancer-free sample data 210 of FIG. 2, the non-cancer region sample data 211 of FIG. 2, and/or the cancer sample data 212 of FIG. 2 different or separate from the data used for the feature selection.

### Approaches to Applying the Trained Model

FIG. 7B shows a flow chart of an example method 750 of operating a computing system (e.g., the system 100 including the source device 152 and/or the processing system 102 as illustrated in FIG. 1B) to analyze or test a patient’s unbounded sample in accordance with one or more implementations of the present technology. The method 750 can further include collecting and isolating the DNA data of a patient. The resulting targeted DNA data can be provided to the system as input data for analyzing the existence or the likely onset of targeted diseases, such as cancer.

In some implementations, the method 750 can include collection of unbounded samples, such as illustrated at block 752. For example, the collection portion of the method 750 can include obtaining blood samples, saliva samples, cheek swabs, or the like from the targeted patient. The samples can be collected with or without suspicion of cancer, such as for samples collected as a part of routine physical examinations.

The collected unbounded sample can be further processed to isolate one or more targeted components therein as illustrated in block 754. In some implementations, the targeted/isolated component can include leukocytes or white blood cells within the collected blood sample.

At block 756, the DNA can be extracted from the isolated target. Using one or more lab techniques, the targeted component (e.g., the leukocytes) can be broken up, and targeted portions, such as the nucleus, can be further isolated. The DNA can be removed from the isolated result. Additionally, the extracted DNA may be subjected to a cleaning process to increase the purity of the DNA.

At block 758, the extracted DNA may be processed to produce corresponding data, such as the target DNA data 772. For example, the extracted DNA can be sequenced to determine the sequence of bases within the DNA. In some implementations, the sequenced DNA can be based on targeted markers that correspond to the total set or the reduced subset of the usable locations. As a result, the DNA processing can generate target DNA data 772 (e.g., text strings) representative of the DNA sequence of the targeted portion in the unbounded sample.

It has been discovered that the DNA data derived from the leukocytes provide reduced noise parameters, such as other diseases, effects of pathogens or other physiological conditions, and/or mutations unrelated to the development of various cancers. As such, analyzing the DNA data derived from the leukocytes provides increased accuracy in detecting or characterizing the somatic mutations in or throughout the patient body.

In some implementations, the target DNA data 772 can include preprocessed and/or formatted results of the text strings. As an illustrative example, the target DNA data 772 can follow the example analysis template 500 (e.g., a sequence of counts arranged according to an order in the derived phrases, with each count representing a number of matching text strings) to represent the sequenced data (e.g., the text strings). For the formatting/preprocessing, the text strings can be compared against the initial features set 114 of FIG. 1A and/or the select feature set 124 of FIG. 1A. The system 100 (via, e.g., the sourcing device 152 of FIG. 1B, the processing system 102 of FIG. 1B, or both) can generate a set of numbers that are arranged/sequenced to correspond to the set of selected features. Each number in the set can identify the number of times the corresponding feature (unique text string) was found within the patient’s sequenced DNA information. Additionally or alternatively, each number in the set can represent a mathematical combination of the counts for a predetermined grouping of selected features. Accordingly, the system 100 can further reduce the size of the data communicated and/or processed using the ML model. Moreover, the DNA data or the sequence of counts can be preprocessed (e.g., according to a predetermined mathematical formula) to remove various biases (e.g., capture bias) introduced by the preceding steps, such as DNA isolation, DNA extraction, etc.

At block 760, the system 100 (via, e.g., the processing system 102) can analyze the DNA data or the preprocessed result thereof using one or more ML models. For example, the processing system 102 can process the analysis template 500 having values specific to the DNA information corresponding to the patient’s unbounded sample.

Effectively, the analysis can include receiving the formatted DNA data (e.g., the target DNA data 772) as illustrated at block 782. The received target DNA data 772 can represent DNA segments found in the unbounded sample.

At block 786, the system can analyze the mutations represented by the target DNA data 772. In other words, the system 100 can test the target DNA data 772 against the ML model 104 of FIG. 1B, thereby implementing the trained model to compute a cancer signal/score. The system can analyze the mutations by identifying text strings within the target DNA data 772 that match the set of derived phrases (e.g., textual representations of unique mutations, such as indel mutations as described above).

Effectively, the system 100 can identify and quantify/measure the somatic mutations reflected in the target DNA data 772. The system 100 can generate a signal or a score that characterizes the somatic mutations in the target DNA data 772 with respect to one or more types of cancers. In other words, the trained model can be configured to measure overlaps between (1) the somatic mutations found in the patient white blood cells and (2) somatic mutations characteristic (as represented by the derived phrases) of one or more types of cancers. The resulting measure can indicate whether the patient has cancer, whether the patient is without cancer, whether the patient has a specific type of cancer, how close the patient is to the onset of one or more types of cancer, one or more likelihood scores thereof, or a combination thereof.

In some implementations, multiple models can be used to analyze the target DNA data 772. For example, the system 100 can use different models to assess whether the patient has cancer, whether the patient is without cancer, and whether the patient has a specific type of cancer. Also, the system 100 can use region-specific-sample models along (e.g., in parallel or in sequence) with unbounded-sample models.

determining a sequence of counts that have been arranged according to a predetermined sequence of the set of derived phrases, wherein each count in the sequence of counts represents a quantity of text strings within the target DNA data that matched a corresponding derived phrase in the predetermined sequence

Most cancer mutations take years to develop (e.g., 10 years) before the onset of tumorigenesis, even the DNA of the healthy patient is likely to have some signatures of cancer. It has been discovered that using certain targeted components in unbounded samples, such as the leukocytes in patient blood samples, the system 100 can accurately assess the state of such cancer mutations/development. As a result, the system 100 can generate the analysis output that effectively detects the onset of cancer or detects cancers that have yet to cause recognizable symptoms. Moreover, the reduced processing burdens and the capacity to use general non-localized biological samples (e.g., before any suspicion of cancer) described above can provide the capacity to monitor the progress of treatments. In other words, the system can analyze the DNA data for a reversal in the mutation trend or the change in the amount of such cancerous DNA caused by cancer treatments.

At block 762, the system 100 can provide assistance in responding to the findings. For example, the system 100 can provide the analysis results (e.g., the evaluation result 134 of FIG. 1B including the cancer signal) to healthcare professionals and/or the analyzed patients. In some implementations, the system 100 can provide recommendations for additional tests (e.g., biopsies, CT scans, or the like), implement additional analysis (e.g., application of models or other diagnostics specific to physiological locations and/or probable type of cancer) for further details, and/or treatment options. The recommendations may also be for collecting/analyzing certain locations or tumors on the patient body and/or applying cfDNA/ctDNA and/or CTC diagnostic in addition to the analysis using the ML model that has features different from the unbounded model and unique to the cancerous tissue.

Since the system 100 can observe the progress of the cancer treatments at the DNA-level, the system 100 can provide additional/lower-level (e.g., faster responding) view regarding the efficacy of the ongoing or implemented treatment. Such additional insight can provide healthcare professionals the ability to change and update the treatments earlier. Additionally, the observation data can be crowd-sourced and analyzed across other factors (e.g., ethnicity, preexisting conditions, other medications, or the like) to assess/predict the efficacy of treatment options for different patients. Thus, the system 100 can be configured (via, e.g., similarly trained treatment recommendation models) to provide accurate and personalized treatment recommendations.

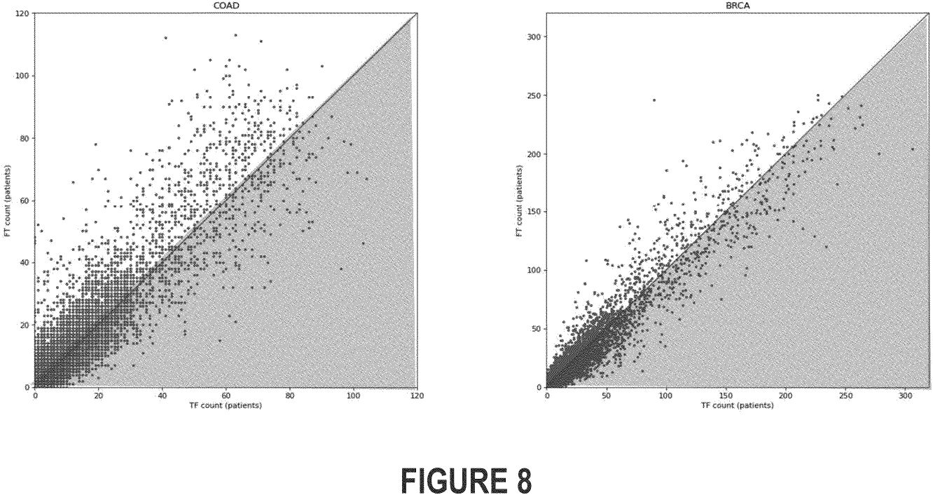

FIG. 8 shows charts illustrating detected mutations in tumor samples and general samples using the usable locations (e.g., the total set and/or the reduced set, such as for the TRSs, the k-mers, and/or the tandem repeat associated k-mers described above) in accordance with one or more embodiments of the present technology. FIG. 8 illustrates the cancer signal in unbounded DNA in comparison to tumor DNA for two example types of cancers. The charts illustrate FT counts in comparison to TF counts by TRS-indels for COAD and BRCA. The scatter plot dots below the diagonal line can represent occurrences when the amount of the targeted subset of TRSs that were found to be mutated in cancer patients’ unbounded samples (e.g., leukocytes) exceeded corresponding amounts found in the tumor tissue. Thus, FIG. 8 illustrates the existence of cancer-characteristic mutations in the unbounded samples. Similar links have been found for other unbounded samples, such as for saliva samples.

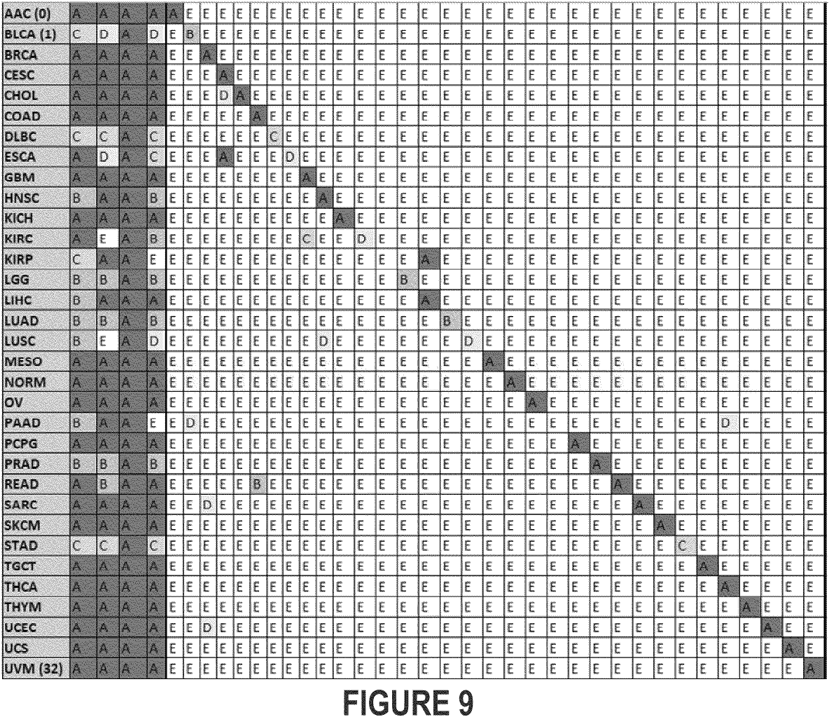

FIG. 9 shows a chart illustrating a matrix of likelihood values output by a model upon being applied to sample DNA information of an example set of patients. This cancerous sample DNA information was obtained from TCGA, and so the health states of those exemplary patients were known. Said another way, it was known which cancer type was assigned to each sampled patient.

In reviewing FIG. 9, there are several items worth mentioning. First, precision, recall, and F1 scores or ratings were produced for each cancer type. Second, the likelihood entries along the diagonal indicate the relative strength of the multiclass model to classify the corresponding cancer type. Ideally, the precision and recall results should be high, with the highest result (e.g., likelihood values or ratings) existing on the diagonal. When the highest likelihood value exists on the diagonal, it can be inferred that predictions of the corresponding cancer type are likely to be accurate. This relationship is generally proportional. As such, the higher the result along the diagonal, the higher the likelihood that predictions for the corresponding cancer type will be accurate. FIG. 9 illustrates the results using letter ratings (e.g., sequentially A, B, C, D, and F with A being the highest or most optimal result). In some embodiments, the letter ratings can correspond to a predetermined range of likelihood values (e.g., A for likelihood values greater than 0.5, B for values between 0.4 and 0.5, etc.) In other embodiments, the output matrix can include the likelihood values. The likelihood values included in each row of the matrix can sum to one.

However, there may also be other non-zero entries that may be interesting as further discussed below. In addition to a satisfactory result (e.g., a calculated number, such as a likelihood value, exceeding a predetermined threshold/range) on the diagonal, the multiclass model should also produce satisfactory results for precision. At a high level, precision indicates how strongly the system is testing for “true positive” and “false positive.” Similarly, the multiclass model should produce satisfactory results for recall. At a high level, recall indicates how strongly the system is testing for “true negative” and “false negative.” When (i) the highest likelihood value exists on the diagonal and (ii) precision and recall are high, it can be inferred that the genetic information provided to the multiclass model as training data is showing a “strong signal” of the corresponding cancer type (and thus, is supported by the various metrics).

Determining whether precision and recall are sufficiently “high” is an important aspect of establishing whether the multiclass model is being properly trained. The determination of whether the value is sufficient may not be static, but instead could be dynamically determined. Accordingly, for precision and recall, a value may be considered “high” if it exceeds a threshold that is representative of a static value per cancer type that can be adjusted based on factors such as cancer type, relationship to other cancers, metastatic nature of a patient’s cancer, medical records, and other biomarkers (e.g., blood level of Prostate-Specific Antigen (PSA) for prostate cancer). Additionally or alternatively, the value may be compared to the signal from the matrix and the likelihood value on the diagonal.

Determining whether the likelihood value on the diagonal is “high” is an important aspect of establishing whether the multiclass model is likely to produce useful outputs (e.g., predictions). The focus is not simply on the absolute magnitude of the likelihood value on the diagonal, but the fact that a “row” will add up to one, so the higher the likelihood value on the diagonal, the stronger the signal is for the corresponding cancer type. Again, the likelihood value should be examined in the context of the metrics mentioned above. Note that other non-zero values may be instructive in some instances, especially when the likelihood value on the diagonal is not particularly strong (e.g., less than 0.5). In particular, these other non-zero values may provide insights through comparison to one another and the precision and recall values.

There may be some cancer types where the precision and recall numbers are low and the highest likelihood value is not on the diagonal (or the likelihood value on the diagonal is not significantly greater than at least one other likelihood value). In such a scenario, it can be inferred that predictions of that cancer type will not be as clear based on the relative weakness of the likelihood value on the diagonal. The likelihood value on the diagonal may be considered “weak” if (i) the highest likelihood value is not located on the diagonal, (ii) there is not a clear highest likelihood value in the row, or (iii) even if the highest likelihood value is on the diagonal, the difference between the highest likelihood value and the next highest likelihood value is small (e.g., less than 0.1 or 0.2). Predictions for these cancer types are not as clear as those predictions produced for cancer types for which the highest likelihood value is on the diagonal. While the predictions may not be clear, the system could still look at the other non-zero values along the same row for further information to continue additional analysis. It is worth noting that when the highest likelihood value is not on the diagonal, the precision and recall values are also likely to be low (e.g., below 0.5 or 50 percent).

When this occurs, the system can further investigate why the genetic information provided to the multiclass model as input is not showing a “strong signal” of a given cancer type (and thus, is not supported as evidenced by the low values for precision and recall). Once again, the determination of whether a value for precision or recall is “low” may not be static, but instead could be dynamically determined. Accordingly, for precision and recall, a value may be considered “low” if it does not exceed a threshold that is representative of a static value per cancer type that can be adjusted based on factors such as cancer type, relationship to other cancers, metastatic nature of a patient’s cancer, medical records, and other biomarkers (e.g., blood level of PSA for prostate cancer). Additionally or alternatively, the value may be compared to the signal from the matrix and the likelihood value on the diagonal.

To determine whether the likelihood value on the diagonal is “low,” the system may not simply examine the absolute magnitude of the likelihood value on the diagonal. Because a “row” will add up to one, the higher the likelihood value on the diagonal, the stronger the signal is for the corresponding cancer type, though the determination of whether the likelihood value is “low” may still be factor based. Again, the likelihood value should be examined in the context of the metrics mentioned above

Note that the terms “low” and “high” refer to numeric value or a corresponding rating, rather than the informative value of a likelihood value or a metric value (e.g., for precision or recall). Even if a likelihood value is “low,” significant insight into health can be gained through analysis of the low likelihood value in the context of other non-zero likelihood values.

### Computing System

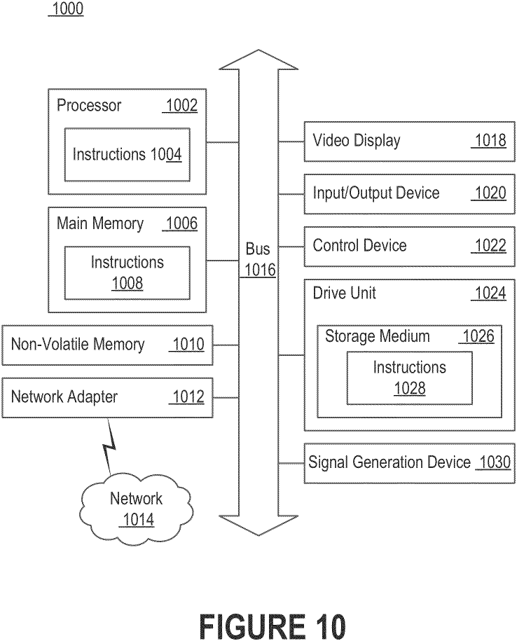

FIG. 10 is a block diagram illustrating an example of a system 1000 (e.g., the computing system 100 or a portion thereof, such as the processing system 102) in accordance with one or more implementations of the present technology. For example, some components of the system 1000 may be hosted on a computing device that includes a mutation analysis mechanism and a refinement mechanism.

The system 1000 may include a processor 1002, main memory 1006, non-volatile memory 1010, network adapter 1012, video display 1018, input/output device 1020, control device 1022 (e.g., a keyboard or pointing device), drive unit 1024 including a storage medium 1026, and signal generation device 1030 that are communicatively connected to a bus 1016. The bus 1016 is illustrated as an abstraction that represents one or more physical buses or point-to-point connections that are connected by appropriate bridges, adapters, or controllers. The bus 1016, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), inter-integrated circuit (I²C) bus, or an Institute of Electrical and Electronics Engineers (IEEE) standard 1394 bus (also referred to as “Firewire”).

While the main memory 1006, non-volatile memory 1010, and storage medium 1026 are shown to be a single medium, the terms “machine-readable medium” and “storage medium” should be taken to include a single medium or multiple media (e.g., a centralized/distributed database and/or associated caches and servers) that store one or more sets of instructions 1028. The terms “machine-readable medium” and “storage medium” shall also be taken to include any medium that is capable of storing, encoding, or carrying a set of instructions for execution by the system 1000.

In general, the routines executed to implement the present technology may be implemented as part of an operating system or a specific application, component, program, object, module, or sequence of instructions (collectively referred to as “computer programs”). The computer programs typically comprise one or more instructions (e.g., instructions 1004, 1008, 1028) set at various times in various memory and storage devices in a computing device. When read and executed by the processors 1002, the instruction(s) cause the system 1000 to perform operations to execute elements involving the various aspects of the present disclosure.

Further examples of machine- and computer-readable media include recordable-type media, such as volatile memory devices and non-volatile memory devices 1010, removable disks, hard disk drives, and optical disks (e.g., Compact Disk Read-Only Memory (CD-ROMS) and Digital Versatile Disks (DVDs)), and transmission-type media, such as digital and analog communication links.

The network adapter 1012 enables the system 1000 to mediate data in a network 1014 with an entity that is external to the system 1000 (e.g., between the processing system 102 can the sourcing device 152) through any communication protocol supported by the system 1000 and the external entity. The network adapter 1012 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### Remarks

The foregoing description of various implementations of the claimed subject matter has been provided for the purposes of illustration and description. It is not intended to be exhaustive or to limit the claimed subject matter to the precise forms disclosed. Many modifications and variations will be apparent to one skilled in the art. Implementations were chosen and described in order to best describe the principles of the invention and its practical applications, thereby enabling those skilled in the relevant art to understand the claimed subject matter, the various implementations, and the various modifications that are suited to the particular uses contemplated.

Although the Detailed Description describes certain implementations and the best mode contemplated, the technology can be practiced in many ways no matter how detailed the Detailed Description appears. Implementations may vary considerably in their details, while still being encompassed by the specification. Particular terminology used when describing certain features or aspects of various implementations should not be taken to imply that the terminology is being redefined herein to be restricted to any specific characteristics, features, or aspects of the technology with which that terminology is associated. In general, the terms used in the following claims should not be construed to limit the technology to the specific implementations disclosed in the specification, unless those terms are explicitly defined herein. Accordingly, the actual scope of the technology encompasses not only the disclosed implementations, but also all equivalent ways of practicing or implementing the present technology.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various implementations is intended to be illustrative, but not limiting, of the scope of the technology as set forth in the following claims.

What is claimed is:

1. A method of developing an artificial intelligence (AI) and/or a machine-learning (ML) model configured to analyze DNA data, the method comprising:
identifying a set of unique segments, each unique segment including a unique repeated text pattern representative of a unique portion within a human genome;
generating a set of expected phrases based on the set of unique segments, wherein each expected phrase includes a unique combination of flanking texts before, after, or both relative to the corresponding unique segment;
generating a set of derived phrases for each expected phrase based on adjusting one or more texts therein, wherein each derived phrase includes a text string representative of a unique somatic insert-deletion (indel) variant of the expected phrase;
deriving a set of selected phrases based on analyzing unbounded sample data using the set of expected phrases, the set of derived phrases, or a combination thereof,
wherein the unbounded sample data includes textual representations of portions of DNA found within unbounded biological samples that have been collected from a region on bodies of previous patients confirmed to have a type of cancer,
wherein the collection region is different from a location affected by the type of cancer; and
developing the ML model based on the set of selected phrases, wherein the ML model is trained and configured to compute a cancer signal based on analyzing an evaluation target,
wherein the evaluation target includes text-based representations of portions of DNA found within a subsequent unbounded sample from an evaluated patient,
wherein the cancer signal represents (1) a likelihood that a corresponding patient has developed the type of cancer or (2) a development status at least leading up to or recovering from onset of the type of cancer.

2. The method of claim 1, wherein:
the set of selected phrases includes text strings indicative of multiple types of cancer; and
the ML model is trained and configured to compute the cancer signal corresponding to one or more of the multiple types of cancer.

3. The method of claim 1, wherein:
the unbounded sample data represents the portions of DNA found within leukocytes of the previous patients; and
the ML model is trained and configured to compute the cancer signal based on the evaluation target representative of the portions of DNA found within leukocytes of the evaluated patient.

4. The method of claim 1, wherein:
the unbounded sample data represents the portions of DNA found within saliva or cheek swab of the previous patients; and
the ML model is trained and configured to compute the cancer signal based on the evaluation target representative of the portions of DNA found within saliva or cheek swab of the evaluated patient.

5. The method of claim 1, wherein the set of selected phrases is deriving based on analyzing the unbounded sample data of the previous patients directly for indications of the type of cancer instead of use as control in analyzing other DNA data derived from cancerous regions or tissues of the previous patients.

6. A system for analyzing patient DNA data using one or more machine-learning (ML) models, the system comprising:
at least one processor; and
at least one memory coupled to the at least one processor and including processor instructions that, when executed by the at least one processor, perform operations including --
receiving a target DNA data representative of DNA in an unbounded biological sample collected from region on a body of a patient, wherein the collection region of the unbounded biological sample is unrelated to a specific location affected by a type of cancer;
computing a cancer signal based on analyzing the target DNA data using one or more trained ML models, wherein the cancer signal represents (1) a likelihood that a corresponding patient has developed the type of cancer or (2) a development status at least leading up to or recovering from onset of the type of cancer; and
providing a medical response assistance based on the cancer signal.

7. The system of claim 6, wherein the target DNA data represents the DNA found within a blood sample of the patient.

8. The system of claim 7, wherein the target DNA data represents the DNA found within leukocytes in the blood sample.

9. The system of claim 6, wherein the target DNA data represents the DNA found within a saliva sample or a cheek swab sample of the patient.

10. The system of claim 6, wherein the cancer signal is computed based on identifying text strings within the target DNA data that match a set of derived phrases that each represent a unique mutation of a unique portion of human genome, wherein the unique portion is represented by a unique repeated text pattern corresponding to the unique portion.

11. The system of claim 10, wherein the cancer signal represents a degree of conformity or overlap between (1) somatic mutations reflected in the target DNA data and (2) somatic mutations characteristically present in unbounded samples collected from patients diagnosed to have the type of cancer.

12. The system of claim 11, wherein the cancer signal is computed based on identifying the text strings within the target DNA data that match the set of derived phrases representative of insert-deletion (indel) mutations in the unique repeated text pattern.

13. The system of claim 6, wherein the cancer signal is computed based on:
determining a sequence of counts that have been arranged according to a predetermined sequence of the set of derived phrases, wherein each count in the sequence of counts represents a quantity of text strings within the target DNA data that matched a corresponding derived phrase in the predetermined sequence; and
computing the cancer signal based on analyzing the sequence of counts or a computational derivative thereof using the ML model.

14. The system of claim 6, wherein providing the medical response assistance includes characterizing a response to a cancer treatment along with providing the cancer signal.

15. The system of claim 6, wherein:
the one or more ML models are configured to screen for multiple types of cancers based on the target DNA data derived from the unbounded biological sample;
the target DNA data is representative of the DNA in the unbounded biological sample collected from the region unrelated to specific locations affected by the multiple types of cancer;
the computed cancer signal represents likelihood values associated with the multiple types of cancers; and
providing the medical response assistance includes identifying one or more subsequent tests specific to one or more types of cancers having corresponding likelihood values exceeding a predetermined threshold.

16. A method of analyzing patient DNA data using one or more machine-learning (ML) models, the method comprising:
receiving a target DNA data representative of DNA in an unbounded biological sample collected from region on a body of a patient, wherein the collection region of the unbounded biological sample is unrelated to a specific location affected by a type of cancer; and
computing a cancer signal based on analyzing the target DNA data using one or more trained ML models,
wherein analyzing includes identifying text strings within the target DNA data that match a set of derived phrases that each represent a unique somatic mutation of a unique portion of human genome, the unique portion represented by a repeated text pattern unique to the corresponding portion,
wherein the set of derived phrases includes at least one phrase that represents a biomarker unique to the unbounded sample and at least partially indicative of the type of cancer, and
wherein the cancer signal represents (1) a likelihood that a corresponding patient has developed the type of cancer or (2) a development status at least leading up to or recovering from onset of the type of cancer.

17. The method of claim 16, wherein computing the cancer signal includes identifying the text strings within the target DNA data that match textual representations of insert-deletion somatic mutations in the repeated text pattern.

18. The method of claim 15, wherein the received target DNA data represents the DNA data derived from leukocytes or saliva collected from the patient.

19. The method of claim 15, wherein computing the cancer signal includes:
determining a sequence of counts that have been arranged according to a predetermined sequence of the set of derived phrases, wherein each count in the sequence of counts represents a quantity of text strings within the target DNA data that matched a corresponding derived phrase in the predetermined sequence; and
computing the cancer signal based on analyzing the sequence of counts or a computational derivative thereof using the ML model.

20. The method of claim 15, wherein:
the one or more ML models are configured to screen for multiple types of cancers based on the target DNA data derived from the unbounded biological sample;
the target DNA data is representative of the DNA in the unbounded biological sample collected from the region unrelated to specific locations affected by the multiple types of cancer;
the computed cancer signal represents likelihood values associated with the multiple types of cancers; and
the method further comprising:
providing assistance in a health response when the likelihood values exceed a predetermined threshold for the type of cancer.
